(19) United States
(12) Patent Application Publication
(10) Pub. No.: US 2003/0147909 A1
(43) Pub. Date: Aug. 7, 2003

(54) Highly immunogenic protein against the intracelluar pathogen agent Piscirickettsia salmonis, which affects salmon culture, amino acid and nucleic acid sequences of said protein and its application in the development of useful methods for the prevention and diagnosis of diseases caused by said pathogen

(75) Inventor: Sergio Hernan Marshall Gonzalez, Valparaiso (CL)

Correspondence Address: MICHAEL BEST & FRIEDRICH, LLP, 100 E WISCONSIN AVENUE, MILWAUKEE, WI, 53202, US

(73) Assignee: Universidad Catoolica de Valparaiso, Valparaiso (CL)

(21) Appl. No.: 10/228,167

(22) Filed: Aug. 26, 2002

(30) Foreign Application Priority Data
Aug. 27, 2001 (CL) 2086-2001

(51) Int. Cl. A61K 39/02; C07K 14/195; C12P 21/02; C12N 1/21; C07H 21/04; C12N 15/74

(52) U.S. Cl. 424/190.1; 435/252.3; 435/320.1; 435/69.3; 530/350; 536/23.7

(57) ABSTRACT

The present invention relates to processes for purifying a highly immunogenic protein derived from naturally infected fish tissues with the intracellular bacterium agent Piscirickettsia salmonis; said novel protein is named CHAP, and is further characterized by its amino acid and nucleic acid sequence which are in turn useful for obtaining the protein full length through genetic engineering.; Bidirectional DNA sequencing of chap revealed that the gene contained a single open reading frame encoding 545 amino acid residues with a predicted molecular mass of 58.5 and 4.9 isoelectric point.

This inmunodominant antigen is intended for use in vaccines against Piscirickettsia salmonis who affects salmonidae species such as coho and atlantic salmons and rainbow trout.

### FIELD OF THE INVENTION

This invention is in general found in the field of molecular biology and more particularly in the area of biotechnology, inasmuch as in its main aspect it describes the form to obtain useful tools for the prevention and diagnosis of the disease caused by Piscirickettsia salmonis; said tools are obtained from a sequence of nucleic acid and a sequence of amino acids.

### BACKGROUND OF THE INVENTION

Breeding of live species under captivity to produce meat destined to direct human consumption—as is the case with salmon culture—represents an activity which is submitted to increasing demand by the consumers and requires high quality standards, an aspect which is decisive for competing and remaining in the domestic and international markets.

The biological subjects which are relevant for the production of salmonidae species have basically been focussed on the fish reproduction, diseases and feeding. Some aspects obtain almost immediate solutions devised by the producing companies, while others require both basic and advanced scientific research.

Chile is at present the second world producer in the salmonidae market with a production and total exportation of approximately 240,000 ton of salmon. An aspect of the culture which is essential for the quality control of the final product and the companies profitability corresponds to the diseases which often attack the fish population in a heap up condition. In view of the importance of this market in our country, the solutions for the combat of bacterial and viral agents having a strong negative impact on the aquaculture should address the discovery of preventive treatments, such as vaccines.

The Salmon Rickettsial Syndrome (“SRS”) or piscirickettsiosis was described early in 1989 and since then it has caused significant moralities in salmonid aquaculture. Piscirickettsia salmonis the causative agent of this syndrome, produces the most important disease affecting the domestic salmon producing system and is translated into the pathology that induces the greatest economic losses in Chile. Data supplied by diverse companies and domestic entities indicate that the losses resulting from this microorganism would reach figures close to US$ 80,000,000, which doubtless reflect the importance of this bacteria as a pathogen agent.

The comprehensive control of diseases in salmon contemplates a series of measures that should be jointly applied. This involves monitoring, fast and accurate diagnose techniques, alternate use or use for short periods of the culture places, a suitable infrastructure offering genetic improvement, optimum hygiene and sanitation of the facilities; nutritional quality of the food, adequate feeding systems, careful handling of the fish, ova with a safe origin and a continuous technical capability. Furthermore, by way of important preventive measures against infection, the market offers techniques and products, such as immunostimulants, hygiene prophylaxis, antibiotics and comprehensive controls of salmon diseases, with special emphasis on the support to the fish defensive system based on vaccines.

The immunostimulant substances are represented by glucanes, vitamin C and biogenic stimulators. Immunostimulants are only recently being tested, and for this reason there is no ready market for them. Both the hygiene prophylaxis and the diseases comprehensive control systems re a substitute based on prevention-oriented actions, but they do not guarantee to stop the fish contagion by bacteria and virus.

The only set of concrete products for the curative and non-preventive control of diseases always applied to the salmonidae culture corresponds to antibiotics, i.e., oxolynic acid, eryhromycin, flumequine and oxitetracycline.

The hygiene prophylaxis corresponding to measures of a hygiene-sanitary nature throughout the process chain of salmon production is based on hygienic measures and uses adequate and disinfectant procedures which have a proven action against the pathogens, in addition to a control carried out through a healthy ova supply.

A National Health Plan elaborated by the Salmon Technological Institute has been implemented in Chile, which is oriented to the normalization and supervision of sanitary handling practices, and to obtain and organize the information provided by the industry in areas which are sensitive to the companies productivity. Its action lines are the surveillance, prevention, diagnosis and diseases control.

All the above mentioned substitute products are insufficient to ensure the eradication of Piscirickettsia salmonis as an obligate intracellular rickettsial pathogen because the failure of them to reach effective levels—for the intracellular location of the infection as well as of the agent The inconsistent outcome of treatment with antimicrobials has encouraged research into the development of vaccines. The sole viable option consists in the preventive management of diseases by means of vaccines, a situation which has been demonstrated by the strategy involving the eradication of the use of antibiotics and other products applied at a domestic level in Norway, whereby their full substitution with vaccines was generated.

In general, vaccines offer the advantage of being a disease preventive management, whereby they would substitute the use of antibiotics and not vice versa, and further present the advantages of a definite efficiency, a considerable and verified costs reduction, a longer duration and real preventive capacity, thus additionally overcoming all the disadvantages inherent in the use of antibiotics.

The market currently offers vaccines for Piscirickettsia salmonis. Thus, under the commercial name “Ricketvac”® is elaborated with Piscirickettsia salmonis isolates duly characterized and titrated, which are obtain in the field, propagated in cell cultures and rendered inactive with formaldehyde.

The fact of its dependence from the full bacteria propagation in cell cultures implies the counting with rigorous sterility conditions and quality controls, which factors complicate the management of its production at the industrial level. Additionally, the fact that the pathogen agent remains intracellular most of the times and surrounded me membrane-type vesicles renders this classical vaccine completely innefective

According to the previous art, which show difficulties in the mass production of the vaccines from isolates of the bacteria as well as a non efficient protection. one goal of the present invention consists in the purification of a highly immunogenic protein derived from the intracellular pathogen Piscirickettsia salmonis. to promote ideally both humoral and cellular responses of the fish against the agent.

Another object of the present invention is to characterize said protein through its amino acid and nucleotides sequence; said sequences will in turn be useful for obtaining the full length-protein by means of genetic engineering.

A very important purpose of the present invention is the development of a vaccine for preventing the infection of salmonidae species, exemplified by but not restricted to Coho salmon, Atlantic salmon and rainbow trout by the Piscirickettsia salmonis pathogen.

In view of the foregoing, its objective is to obtain a vaccine from the protein as well as from the latter's DNA.

A further object is to provide an efficient tool for the detection of Piscirickettsia salmonis, which is an important aspect in any comprehensive control program of the disease caused by this pathogen-In order to do so a battery of different anitibodies against the protein have been elicited in different organisms to promote sensitive detection in the field

The vaccine according to the present invention is distinguished by its high effectiveness in preventing salmonidae contagion with Piscirickettsia salmonis, and records much higher levels as compared with those obtained from other products, and without undesirable side effects, such as adherence-, for example.

It may be concluded that there is a wide market for the Piscirickettsia salmonis vaccine, which can compete by far with the existing vaccines, as it will be demonstrated hereinbelow. The vaccines have no real substitutes. On the contrary, this product will substitute the use of antibiotics—chemical compounds that have strategically proposed to eliminate in salmon culture within the shortest possible time.

### DETAILED DESCRIPTION OF THE INVENTION

In an effort to characterize and purify the CHAP protein of Piscirickettsia salmonis, we adopted a combined genomic-proteomic approach based upon the following features,

i) Purification of Piscirickettsia salmonis: from CHSE-214 tissue culture infected cells as close as possible to homogeneity by means of differential centrifugation of shedded bacteria followed by DNase I digestion to get rid of cellular contaminant DNA and purified by - lodixanol density gradient centrifugation—,

ii) Production of rabbit immune sera induced against the purified bacteria—obtained in rabbits as polyclonal antibodies To be used for detection and characterization purposes

iii) - Identification of the best immunogenic protein CHAP from in vivo naturally infected fish as well as from tissue culture induced infection with the pathogenic agent: Differential hydrophobic protein extracts were obtained by a novel procedure from; naturally infected coho salmon fish organs (liver, kidney and brain) as well as from CHSE-214 infected cells. Resulting proteins were analyzed by polyacrilamide gels (SDS-PAGE) uni and bidimensional, followed by isoelectric focusing to determine corresponding isoelectric points of the separated proteins. western blot analysis using the battery of antibodies yielded the most reactive as a surface protein antigen, named CHAP thereafter-

iv) -,- isoelectric point -of the protein turned out to be 4.9 and the protein was blotted from the 2-D gel onto an PVDF membrane to be as pure as possible for sequencing purposes. -The immunoreactive spot was submitted -to microsequencing by the standard well-known Edman degradation procedure. The salmonid immune system humoral and presumably cellular does appear to strongly react to this P. salmonis antigen and render protection to primary and secondary infection . The purified - CHAP - protein has a molecular mass of 58.5 kDa and an isolectric point of 4.9.

Once the peptide secuence (Table 1) were obtained, a Basic Local Alignment Search Tool analysis (BLAST) was performed to search for homologous heterologous proteins that might suggest the potential biological function as well as the type of protein CHAP represented -. The analysis showed that the peptide sequenced -observed distinctive homology although remaining quite specific for P. salmonis with the chaperoning encoded -the groEL genes of Proteobacteria species.

Chaperonins are oligomeric molecular chaperones. They belong to the heat shock protein family (HSP's), which are highly conserved and found in all prokaryotic cells. HSPs are induced by a variety of environmental stresses, such as temperature, inflamation, viral and bacterial infections as well as malignant transformation. GroEL can be adscribed to the HSP60 family of chaperonins, - -best characterized in E.coli and very similar to the 65 kDa antigen of Mycobacterium tuberculosis spp. In both cases they are thought to be immunodominant antigens, which facilitate forlding, unfolding and translocation of polypeptides - allowing the assembly and disassembly of oligomeric protein complexes. Thus the protein family has pivotal roles in normal cell functioning. Chaperonines, as immunodominant antigens. -are so abundantly expressed that they saturate the epitopes of the immune system. - Nonetheless, at the same time as - highly preserved proteins, they are sufficiently divergent in -- their nucleotide sequence to provide group-specificity, meaning that each chaperonin from each phylogenetic group analyzed is by itself different from their counterparts.

The literature abounds with controversy about the roles of molecular chaperones and their role in immunity. This confusion might be due in part to the increasing evidence that molecular chaperones are not simply inert immunogens but can participate in standard immune response activation such as those of lymphocytes. Particularly- chaperonins should be classified as ‘multiplex antigens’ because of their ability to interact with, and activate different cells types.

On the basis of the amino acid sequence - obtained for the P. salmonis GroEL, two degenerated primers B1 and AB4 were designed upon alignment with others groEL gene secuences in order to promote its amplification from the purified P. salmonis genome. These primers cover 70% of the total gene - lengtht PCR was performed using primer sets as indicated in Table 2, under - standarized optimal conditions for cloning. Resulting DNA amplicons were cloned into a TOPO-TA expression vector, Expression and purification of the cloned gene was attained using the PurePro Caulobacter Expression System a new protein production system from Invitrogen based on the bacterium Caulobacter crescentus which delivers the pure protein into the growing media thus simplifying its recovery. -.

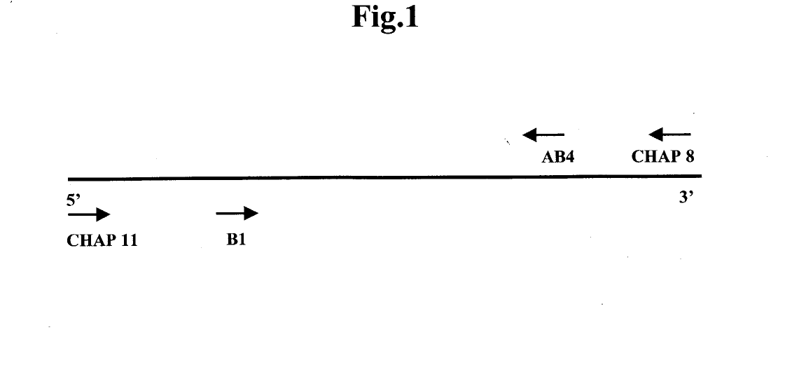

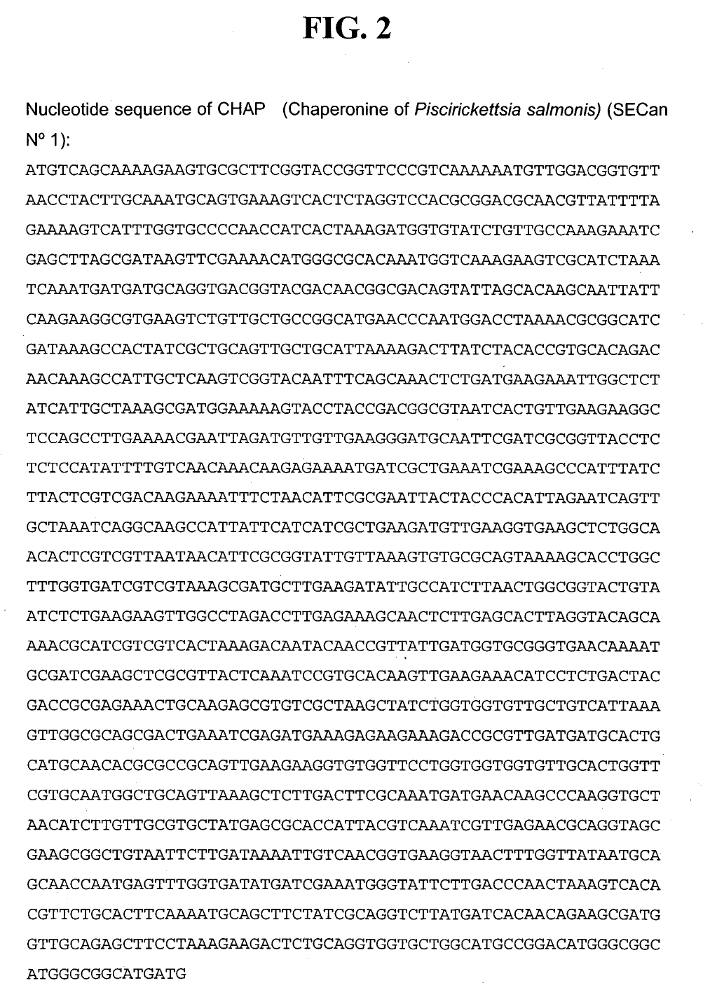

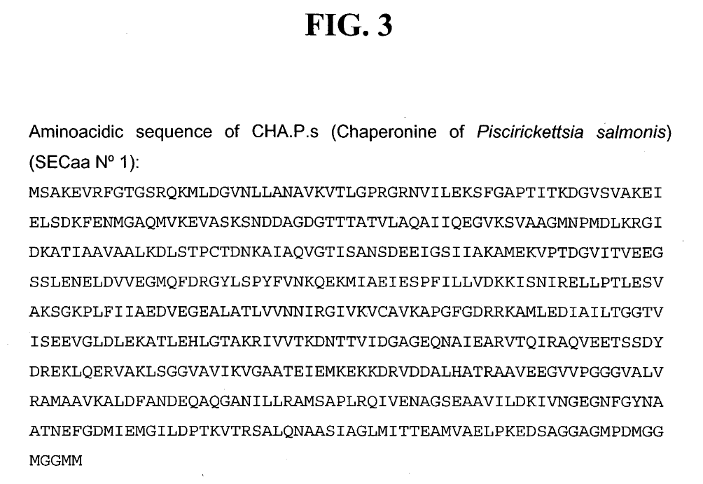

Upon the sequence specificity of the expressed truncated protein (70%) was confirmed -, a new CHAP BLAST alignment was done in order to design degenerated primers set to cover the full length- of said novel P.salmonis protein (Table 2, FIG. 1).

The full length- of CHAP protein amplified under standard conditions with the new primers CHAP11 and CHAP8 was also cloned in the expression vector pCXTOPO-TA. The plasmid carrying the recombinant gene encoding the fusion protein was introduced into Caulobacter crescentus via electroporation. Electrotransformants were selected and grown on expression medium for a further 72- 96 h. The aggregated fusion protein was recovered from the culture medium by sieving through nylon mesh, rinsed and resuspended for the vaccine formulation.

The better description, the more protected we will be.

TABLE 1
Deduced amino acid residues from protein CHAP

| Peptide sequence | Peptide no. |
|---|---|
| SFGAPTITK | 43-51 |
| FENMGAOMVK | 66-75 |
| VAAGMNPMDLKR | 107-118 |
| ELLPTLESVAK | 232-242 |
| AAVEEGVVPGGGVALVR | 405-421 |

TABLE 2

Oligonucleotide primers used for PCR

| | | |
|---|---|---|
| Primer | Sequence (5′ to 3′) | Position^{a} |

| | | |
|---|---|---|
| Forward | ^{b} | |
| CHAP 11 | GGAGATATAAGA\overline{ATG}TCAGCAAAAGAAGTG | 1-18 |
| B1 | GTCHTTCGGYGCDCCRACCATYAC | 126-149 |
| Reverse | | |
| CHAP 8 | CATCATRCCGCCCATKCCRCCCAT | 1612-1635 |
| AB4 | CCRCCYGGWACVACRCCTTCTTC | 1244-1222 |

^{a}Position relative to P. salmonis GroEL genomic sequence described in this petition
^{b}Starting Metionine

1. A protein CHARACTERIZED in that it is provides immunogenicity against Piscirickettsia salmonis pathogen, naturally obtained through the activation of the immune system of fish inoculated with the pathogen.

2. A protein as described in claim 1 CHARACTERIZED in that it has a molecular mass 58.5 kDa and P.I. 4.9.

3. A protein as described in claim 1, CHARACTERIZED in that is corresponds to the sequence of aa. SECaa No. 1. -

4. A protein as described in claim 1, CHARACTERIZED in that it corresponds to the sequence of SEC na No. 1 nucleic acids sequence.

5. A protein as described in claim 1, CHARACTERIZED in that it corresponds to analogues and/or derivatives and/or degenerated sequences of the SEC na No. 1 nucleic acids.

6. A protein homologous to the GroEL protein described in claim 1 derived from another species of bacteria exemplified by but not excluded to E. coli, M. tuberculosis, . . . etc. and its use in a vaccine for fish and others marine organisms.

7. A recombinant DNA molecule, CHARACTERIZED in that it comprises the nucleic acids sequence defined in claims 4 and/or 5 and one or more expression control sequences operatively bonded to the nucleic acid sequence.

8. A unicellular host, CHARACTERIZED in that it is transformed with a DNA recombinant molecule according to claim 6.

9. A protein or a peptide as described in claim 1, CHARACTERIZED in that it is alternatively obtained through the culture of a unicellular host, according to claim 7 and the isolation of said protein.

10. A method of preparation of a vaccine to prevent the infection of fish i.e salmonides exemplified but not restricted to Coho salmon, Atlantic salmon, Rainbow trout or non-salmonides exemplified but not restricted to White seabass, Black seabass, Tilapia with Piscirickettsia salmonis, CHARACTERIZED in that it comprises the mixture of the protein of claim 1 with a pharmaceutically acceptable diluent, excipient or adjuvant.

11. The use of the protein according to claim 1 or a fraction thereof, CHARACTERIZED in that it is destined to the elaboration of a vaccine to prevent the infection of fish i.e salmonides exemplified but not restricted to by Coho salmon, Atlantic salmon, Rainbow trout or non-salmonide exemplified but not restricted to White seabass, Black seabass, Tilapia with the Piscirickettsia salmonis bacteria.

12. A method of preparation of a vaccine to prevent the infection of fish i.e salmonides exemplified but not restricted to by Coho salmon, Atlantic salmon, Rainbow trout or non-salmonide exemplified but not restricted to White seabass, Black seabass, Tilapia with Piscirickettsia salmonis, CHARACTERIZED in that it comprises the steps of mixing the protein of claim 4 with a pharmaceutically acceptable diluent, excipient or adjuvant.

13. A method of preparation of a vaccine to prevent the infection of fish i.e salmonides exemplified but not restricted to by Coho salmon, Atlantic salmon, Rainbow trout or non-salmonide exemplified but not restricted to White seabass, Black seabass, Tilapia, with Piscirickettsia salmonis in combination with antigens protecting against other bacterial and viral diseases in firsh exemplified but not restricted to Aeromonas salmonicida, Vibrio anguillarum, Renibacterium salmoninarum, Yersinia ruckeri, Infectious pancreas necrosis virus, Infectious salmon anemia virus, CHARACTERIZED in that it comprises the steps of mixing the protein of claim 4 or fragments thereof, with other antigens in a pharmaceutically acceptable diluent, excipient or adjuvant.

14. The use of a sequence of nucleic acids as described in claims 4 or 5 or a fraction thereof, CHARACTEIZED in that is destined to the elaboration of a vaccine to prevent infection of fish i.e salmonides exemplified but not restricted to Coho salmon, Atlantic salmon, Rainbow trout or non-salmonides exemplified but not restricted to White seabass, Black seabass, Tilapia with the Piscirickettsia salmonis bacteria.

15. A method for the detection of the Piscirisckettsia salmonis bacteria in a biological sample, CHARACTERIZED in that it comprises the steps of:
(a) isolating the biological sample;
(b) contacting said sample either with (i) the protein of claim 1 or (ii) a DNA probe according to the sequence of claims 4 or 5,
(c) detecting the previous bond of the protein or the DNA probe with the pathogen, if the latter is present in the biological sample.

16. The use of the sequence of nucleic acids as described in claims 4 or 5 or a fraction thereof, CHARACTERIZED in that it is destined to the elaboration of a molecular marker for the diagnosis of the infection of fish i.e salmonides exemplified but not restricted to by Coho salmon, Atlantic salmon, Rainbow trout or non-salmonide exemplified but not restricted to White seabass, Black seabass, Tilapia with the Piscirickettsia salmonis bacteria.
